(19) United States
(12) Patent Application Publication
(10) Pub. No.: US 2007/0135808 A1
(43) Pub. Date: Jun. 14, 2007

(54) Surgical instrument

(75) Inventors: Bernhard Kupferschmid, Emmingen-Liptingen (DE); Dieter Weisshaupt, Immendingen (DE)

Correspondence Address: Lipsitz & McAllister, LLC, 755 MAIN STREET, MONROE, CT, 06468, US

(73) Assignee: AESCULAP AG & Co. KG, Tuttlingen (DE)

(21) Appl. No.: 11/638,685

(22) Filed: Dec. 12, 2006

Related U.S. Application Data
(63) Continuation of application No. PCT/EP05/06715, filed on Jun. 22, 2005.

(30) Foreign Application Priority Data
Jun. 23, 2004 (DE) 10 2004 031 927.8

(51) Int. Cl. A61B 18/04; A61F 7/00; A61F 7/12

(52) U.S. Cl. 606/31; 607/96

(57) ABSTRACT

In the case of a surgical instrument with at least one contact face for body tissue and which is made, at least on the surface, of sterilisable plastic and to enable heat treatment of the body tissue resting on the contact face, it is proposed that on the contact face there are provided track-shaped regions with a metal coating which form a heating element, through which current flows when they are connected to an electric voltage source.

This application is a continuation of international application number PCT/EP2005/006715 filed on Jun. 22, 2005.

The present disclosure relates to the subject matter disclosed in international application PCT/EP2005/006715 of Jun. 22, 2005 and German patent application no. 10 2004 031 927.8 of Jun. 23, 2004, which are incorporated herein by reference in their entirety and for all purposes.

### BACKGROUND OF THE INVENTION

The invention relates to a surgical instrument with at least one contact face for body tissue, which is made of sterilisable plastic at least on the surface.

Plastics are being increasingly employed in the manufacture of surgical instruments, e.g. forceps, gripping or clamping instruments, scissors, retractors, probes and the like. In this case, they replace metals that had been customarily used hitherto for the manufacture of these instruments, in particular also in the region of contact faces, with which these instruments come to rest on body tissue and handle or work on this.

In many cases, heat is used for the treatment of body tissue, in particular to stop bleeding, for coagulation or in an extreme case also to cut through tissue. In order to achieve this, it is known, for example, to arrange electrodes in the region of the contact faces that are connected to the poles of a high-frequency voltage source. This then requires a highly complicated structure of the instrument, in particular in the case of conventional metal instruments, since complex insulation measures have to be provided. Such an application is not possible at all in the case of instruments that are made of plastic in the region of the contact face.

It is an object of the invention to also enable heat to act on the tissue resting on a contact face in a simple manner in the case of a surgical instrument of the generic type.

### SUMMARY OF THE INVENTION

This object is achieved according to the invention with a surgical instrument of the above-described type in that track-shaped regions on the contact face are provided with a metal coating and form a heating element, through which current flows when they are connected to an electric voltage source.

In this way, it is possible, despite the use of plastic as material for the contact face, to cause heating of the adjacent tissue in this region by an electric current flow, with which bleeding can be stopped, for example.

In this case, the track-shaped regions can be shaped in very different ways, and it is favourable if the track-shaped regions are arranged in meander form, so that the contact face can be heated virtually over the entire surface. This can be achieved in particular if the track-shaped regions are distributed over the entire contact face, wherein it is favourable if the contact face is covered substantially uniformly by the track-shaped regions. A uniform heating of the contact face is then obtained when current flows through it.

In a preferred embodiment, it is provided that on the contact face additional track-shaped regions are provided with a metal coating. This provides the possibility of allowing further electrical processes. e.g. measurement processes or also additional treatment processes, to proceed independently of the heat flow in the region of the contact face. These additional track-shaped regions could themselves be heated, so that a two-stage heating would be enabled through the different track systems. However, according to a preferred embodiment, it is provided that the additional track-shaped regions form a temperature sensor, e.g. a resistance sensor, or a feed and discharge line for a temperature sensor arranged in the region of the contact face. In this way, the temperature in the contact area can be measured and fed to an automatic control circuit, which controls the supply of current to the track-shaped regions heating the contact face, for example, so that a specific maximum temperature is not exceeded in this region, e.g. in the order of 80 to 90° C.

According to a preferred embodiment, it is provided that the track-shaped regions of the contact face are connected to terminals, likewise made of a sterilisable plastic, through track-shaped regions, which are provided with a metal coating, of a support, on which the contact face is arranged or held.

The electrically conductive track-shaped regions provided with a metal coating can be produced in different ways, for example, according to a first preferred embodiment it is provided that the contact face is made of two different plastics, namely a metallisable plastic, to which an applied metal coating adheres well, and a non-metallisable plastic, to which an applied metal coating does not adhere, and that the metallisable plastic is coated with metal and forms the track-shaped regions of the contact face.

Such a contact face can be produced in a so-called two-component injection moulding process, wherein the plastics having the different metallisation properties are simultaneously injected into a mould in a specific geometric arrangement.

The plastics can be fundamentally different plastics, e.g. PA 6 (polyamide 6) may be used as metallisable plastic and PA 12 (polyamide 12) as non-metallisable plastic. However, plastics of the same type can also be used, wherein the metallisation properties have been changed by additives, e.g. by added catalysts in one of the two plastics that allow metallisation, whereas the plastic without this catalyst does not provide any adhesion for a metal coating.

In another preferred embodiment, the contact face is made from a uniform plastic material, which, however, has a pretreated surface in the track-shaped regions, which is different from that in the remaining portion of the contact face and which has a good adhesiveness for a metal coating, whereas the metal coating does not adhere in the remaining portion of the contact face. Such a surface treatment can be achieved by means of a so-called hot stamping process, for example, or by so-called direct laser structuring. In a hot stamping process, a suitable metal foil is pressed onto the thermoplastic plastic under pressure and heat. The foil is stamped out during the stamping process and fused with the plastic. Suitable foils in this case are foils with a thickness of about 18 to 100 μm.

In direct laser structuring, the surface of the plastic material of the contact face is treated with laser radiation, i.e. only in the region that is to be provided with a metal layer. This can be, for example, an irradiation in the uv range using an Nd:YAG laser, which emits a radiation at a wavelength of 355 nm by means of special non-linear crystals. The focus diameter of this radiation can lie in the range of between 40 and 50 μm, so that extremely narrow regions can be structured. In direct laser structuring plastic with embedded metal nuclei can be used, for example, and the metal nuclei are applied to the surface by the laser irradiation in regions and can assist the deposition of metal layers there. The surface regions structured by laser in this way can be metallised by chemical depositions without external current, e.g. by using layer systems of copper, nickel and/or gold.

The processes outlined above for applying a metal layer all use a technology referred to as “moulded interconnect devices (MID)”, in which electrically conductive regions can thus be applied to plastic material, in particular in conductor track form.

It is particularly advantageous if the plastic used is a thermoplastic, this simplifying the shaping of the instrument parts having the contact face.

The contact face is arranged, for example, on the free end of forceps or at the mouth part of a gripping or clamping instrument. However, other applications for instruments that usually have contact faces for the handling and/or treatment of body tissue are also possible.

In particular, a respective contact face with metal-coated track-shaped regions can be arranged on a surgical instrument on relatively movable tools of the surgical instrument, e.g. on both mouth parts of a clamping instrument.

The following description of preferred embodiments of the invention serves to provide more detailed explanation in association with the drawing.

### BRIEF DESCRIPTION OF THE DRAWINGS

FIG. 1 is a perspective view of a tubular shaft instrument with two mouth parts that are movable relatively to one another;

FIG. 2 is an enlarged detail view of the mouth parts of the instrument of FIG. 1 in region A, and

FIG. 3 is a perspective view of a forceps-type instrument with a heated contact face at the free end of a leg of the forceps.

### DETAILED DESCRIPTION OF THE INVENTION

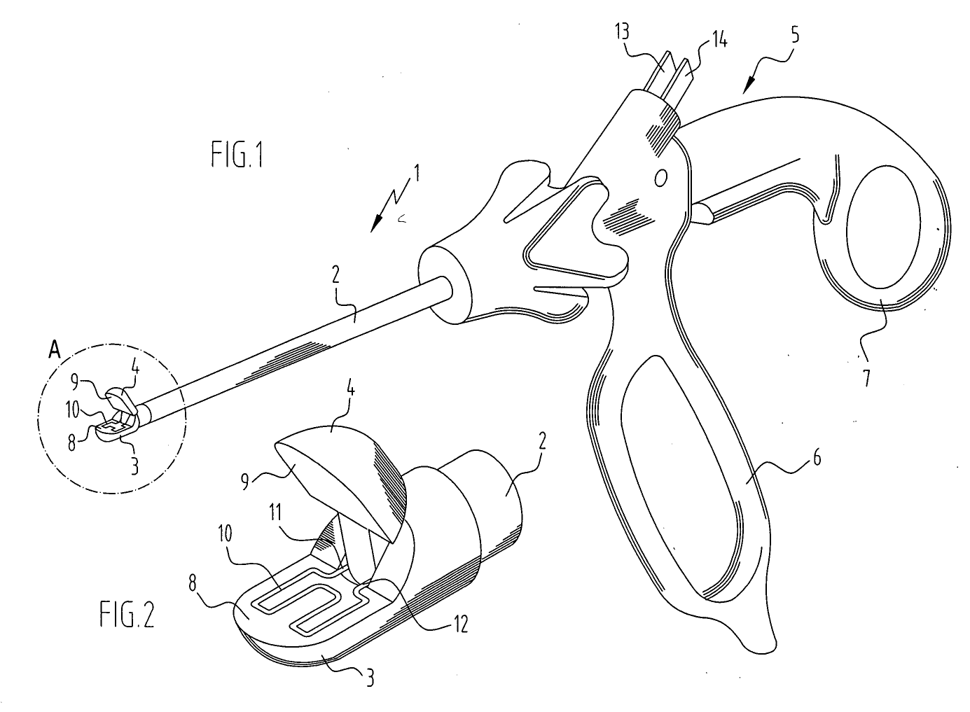

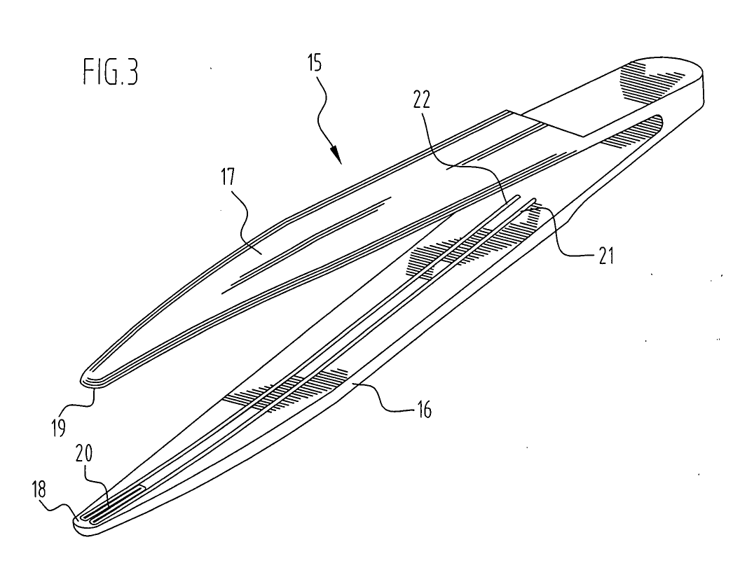

The surgical instrument 1 shown in FIG. 1 is a tubular shaft instrument with an elongated shaft 2, at the distal end of which a fixed mouth part 3 and a mouth part 4, which is able to pivot in relation to this and is itself held on a grip part 5, are arranged. A pivoting branch 7 is able to pivot on the grip part 5 relative to a fixed branch 6 and as a result of this pivoting movement the pivoting mouth part 4 is pivoted by means of a transmission mechanism, which extends through the shaft 2 and is not shown in the drawing, between an open position and a closed position. In the representation in FIGS. 1 and 2, the mouth part 4 is respectively located in the open position, in which it is pivoted away from the fixed mouth part 3, and by pivoting branch 7 towards branch 6 the two mouth parts 3, 4 are brought closer to one another and thus move into the closed position.

On their sides facing one another, the two mouth parts 3, 4 bear flat contact faces 8, 9, which run parallel to one another when the mouth parts 3, 4 are in closed position and either abut flat against one another or stand slightly apart facing one another.

The fixed mouth part 3 is made of a thermoplastic plastic and in the region of its contact faces 8 bears conductor tracks 10, which run in meander shape on these and are distributed over the entire contact face 8 and which form two terminals 11, 12 in the region of the shaft 2. The terminals 11, 12 run via further conductor tracks in the interior of the shaft 2 in a manner not shown to electrical contacts 13, 14 on the grip part 5. There, connection pieces leading to a voltage source can be attached, thus enabling the conductor track 10 overall to be connected into a power circuit that is fed by a voltage source.

When current is passed through, the individual sections of the conductor track 10 are heated, so that heating is distributed over the entire contact face 8, and in this way tissue resting on the contact face 8 can be subjected to heat, as may be necessary to stop bleeding, for coagulation etc.

The conductor tracks 10 are applied to the contact face 8 using one of the techniques outlined above, whether by forming the contact face from different plastics, of which one is metallisable and one is non-metallisable, or by means of a hot stamping process, or by direct laser structuring and subsequent metallisation without external current of the regions of the contact face that have been structured in this way and possibly been cleaned beforehand.

The spatial arrangement of the conductor track 10 can be selected from many different arrangements. In the shown embodiment meandering sections are provided and spiral-shaped arrangements or other configurations of the fitting of the contact face can also be selected. In addition, it is possible to install several heating systems on the contact face 8, so that a stepped heating is possible, and finally additional conductor tracks can be provided, which are not shown in the drawing and which serve to measure the temperature in the region of the contact face. This measurement can occur either by determining the resistance of these additional conductor tracks, which is dependent on the temperature, or by connecting these additional conductor tracks to a temperature sensor arranged in the interior of the mouth part in the direct vicinity of the contact face. This enables the temperature in the contact face region to be controlled, so that in particular unwanted temperature peaks can be prevented.

In the practical example shown in FIGS. 1 and 2, only the fixed contact face 8 can be heated in this manner, and in principle a similar configuration can also be provided in the case of the movable mouth part 4, but this can also be configured without any conductor tracks. It may be favourable to form the opposite mouth part completely from metal, so that this mouth part assists in making the heat development through the opposite mouth part more uniform when tissue is held between the two mouth parts.

FIG. 3 shows forceps 15 with two branches 16, 17, which can be pivoted elastically relative to one another, as a further practical example of a surgical instrument. A contact face 18, which is oriented towards the opposite branch and is also provided with a meandering conductor track 20 like the contact face 8 of the instrument 1, is provided at the free end of each branch. This conductor track is connected to terminals 21, 22, which are arranged on the surface of the branch 16 bearing the contact face 18 and can be connected to the poles of an external voltage source in a manner not evident from the drawing. The forceps are made of thermoplastic plastic and the conductor track 20 is applied in the same manner as that explained on the basis of instrument 1.

Additional conductor tracks can also be provided for temperature determination in this instrument and it is also possible to provide a contact face 19 provided with heated conductor tracks opposite the other branch of the contact face 18.

1. Surgical instrument with at least one contact face for body tissue, which is made of sterilisable plastic at least on the surface, wherein on the contact face there are provided track-shaped regions with a metal coating which form a heating element, through which current flows when they are connected to an electric voltage source.

2. Instrument according to claim 1, wherein the track-shaped regions are arranged in meander form.

3. Instrument according to claim 1, wherein the track-shaped regions are distributed over the entire contact face.

4. Instrument according to claim 3, wherein the contact face is covered substantially uniformly by the track-shaped regions.

5. Instrument according to claim 1, wherein on the contact face additional track-shaped regions are provided with a metal coating.

6. Instrument according to claim 5, wherein the additional track-shaped regions form a temperature sensor.

7. Instrument according to claim 5, wherein the additional track-shaped regions form a feed and discharge line for a temperature sensor arranged in the region of the contact face.

8. Instrument according to claim 1, wherein the track-shaped regions of the contact face are connected to terminals, which also consist of track-shaped regions, provided with a metal coating, of a support of a sterilisable plastic and on which the contact face is arranged or held.

9. Instrument according to claim 5, wherein the track-shaped regions of the contact face are connected to terminals, which also consist of track-shaped regions, provided with a metal coating, of a support of a sterilisable plastic and on which the contact face is arranged or held.

10. Instrument according to claim 1, wherein the contact face is made of two different plastics, namely a metallisable plastic, to which an applied metal coating adheres well, and a non-metallisable plastic, to which an applied metal coating does not adhere, and the metallisable plastic is coated with metal and forms the track-shaped regions of the contact face.

11. Instrument according to claim 10, wherein one of the two plastics contains a catalyst substance and the other does not, and that the two plastics only differ through the content of this catalyst substance.

12. Instrument according to claim 5, wherein the contact face is made of two different plastics, namely a metallisable plastic, to which an applied metal coating adheres well, and a non-metallisable plastic, to which an applied metal coating does not adhere, and the metallisable plastic is coated with metal and forms the track-shaped regions of the contact face.

13. Instrument according to claim 12, wherein one of the two plastics contains a catalyst substance and the other does not, and that the two plastics only differ through the content of this catalyst substance.

14. Instrument according to claim 1, wherein the plastic of the contact face in the track-shaped regions has a pretreated surface, which is different from that in the remaining portion of the contact face and which has a good adhesiveness for a metal coating, whereas the metal coating does not adhere in the remaining portion of the contact face.

15. Instrument according to claim 14, wherein the surface in the track-shaped regions is hot-stamped.

16. Instrument according to claim 14, wherein the surface in the track-shaped regions is directly structured by laser.

17. Instrument according to claim 5, wherein the plastic of the contact face in the track-shaped regions has a pretreated surface, which is different from that in the remaining portion of the contact face and which has a good adhesiveness for a metal coating, whereas the metal coating does not adhere in the remaining portion of the contact face.

18. Instrument according to claim 17, wherein the surface in the track-shaped regions is hot-stamped.

19. Instrument according to claim 18, wherein the surface in the track-shaped regions is directly structured by laser.

20. Instrument according to claim 1, wherein the plastic is a thermoplastic.

21. Instrument according to claim 1, wherein the contact face is arranged on the free end of forceps.

22. Instrument according to claim 1, wherein the contact face is arranged at the mouth part of a gripping instrument.

23. Instrument according to claim 1, wherein a respective contact face with metal-coated track-shaped regions is arranged on relatively movable tools of the surgical instrument.
